(19) United States
(12) Patent Application Publication
(10) Pub. No.: US 2019/0298246 A1
(43) Pub. Date: Oct. 3, 2019

(54) APPARATUS AND METHOD OF CONDUCTING MEDICAL EVALUATION OF ADD/ADHD

(71) Applicant: Obsevera, Inc., Gibsonia, PA (US)

(72) Inventors: Brian J. Lunt, Gibsonia, PA (US); Craig B. Liden, Gibsonia, PA (US)

(21) Appl. No.: 16/374,793

(22) Filed: Apr. 4, 2019

Related U.S. Application Data
(63) Continuation of application No. 15/185,107, filed on Jun. 17, 2016, now abandoned.
(60) Provisional application No. 62/180,739, filed on Jun. 17, 2015.

(51) Int. Cl. A61B 5/16; G16H 50/20; G06F 3/16; G09B 5/12; G16H 30/20; G09B 7/077

(52) U.S. Cl. CPC A61B 5/168; G16H 50/20; G06F 3/167; A61B 3/113; G16H 30/20; G09B 7/077; G09B 5/125

(57) ABSTRACT

A diagnostic method and apparatus facilitating a test for diagnosing attention deficit hyperactivity disorder (ADD/ADHD) in a person is disclosed, which in many embodiments includes a visual problem solving test involving distinctive feature analysis. It facilitates making objective observations of multiple components of attention and executive functioning. This test in some embodiments consists of a 24 individual test items with plates of geometric faces. The test requires the subjects to select two identical faces out a field of faces that progressively increase in number and complexity. The geometric faces could instead be graphic faces, stick figures, geometric shapes or other visual stimuli in other embodiments. Correct/incorrect scores and response times are compared to normative data and coupled with structured behavioral observations to provide objective evidence of the subject's attentional and executive functioning status.

### CROSS-REFERENCE TO RELATED APPLICATION

This application is related to and is a continuation application of U.S. patent application Ser. No. 15/185,107, entitled “Apparatus and Method of Conducting Medical Evaluation of ADD/ADHD” which was filed Jun. 17, 2016 which claims priority from U.S. Provisional Patent Application No. 62/180,739, entitled “Apparatus and Method of Conducting Medical Evaluation of ADD/ADHD” which was filed Jun. 17, 2015 and are hereby incorporated by reference in their entirety.

### SUMMARY OF INVENTION

The present invention is directed to a diagnostic method and apparatus for screening and assisting in diagnosing attention deficit hyperactivity disorder (ADD/ADHD) in a person. For the purpose of clarity, “subject” and “user” are used throughout this description interchangeable. In many embodiments the invention includes a visual problem solving test involving distinctive feature analysis. It facilitates making objective observations of multiple components of attention and executive functioning. To date, it has been used primarily in evaluating individuals suspected of and/or having attention deficit disorder (ADD/ADHD), however additional uses will likely be found by those skilled in the art. The test of the present invention may be administered in a clinical, school, or institutional setting with the assistance of healthcare professionals, teachers, or other persons. The test of the present invention may also be self-administered by the user or test taker.

This test in some embodiments consists of a 24 individual test items with plates of geometric faces. The test requires the subjects to select two identical faces out a field of faces that progressively increase in number and complexity. The geometric faces could instead be graphic faces, stick figures, geometric shapes or other visual stimuli in other embodiments. The test in the 24 item embodiments takes about ten minutes to complete. Correct/incorrect scores and response times are compared to normative data and coupled with structured behavioral observations to provide objective evidence of the subject's attentional and executive functioning status.

The test items could be contained in a test administration booklet or more preferably within a software application that can be administered on electronic devices (e.g. smartphones, tablet computers, or dual-touch computers). U.S. Pat. No. 7,479,949 entitled “Touch screen device, method, and graphical user interface for determining commands by applying heuristics”, which is incorporated herein by reference, describes a device that could be configured via software application to practice a number of embodiments of the present invention.

The test generates objective outcome data including correctness/incorrectness of responses and response times for individual test items and various groupings of items. This data can be correlated with various physiological parameters that are simultaneously recorded during performance of the test/s (e.g. heart rate, galvanic skin resistance, eye tracking movements, facial analysis of emotional or behavioral state, etc.). In addition, when administered by an examiner or when an examiner views a recorded video of the subject taking the test, the examiner completes a checklist of structured behavioral observations made during testing for additional data. It is envisioned this checklist could be completed autonomously via video analysis software using video captured of the subject taking the test/s. For example, software could analyze the facial expressions made by the subject during the test and be able to correlate those facial expressions with specific items of the test. These facial expressions can be used to evidence or even determine the user's mood, emotional or behavioral state.

The present invention provides objective data to help more accurately screen and assist in diagnosing ADD/ADHD and related executive dysfunctions and, thereby, reduce both over and under diagnosis of this common problem. The present invention also provides an objective means to identify the proper medication and dosage regimens for ADD/ADHD individuals requiring medical treatment, thereby, enhancing treatment efficacy, compliance and safety. The present invention provides a means to objectively monitor the evolution of genetically-based ADD/ADHD symptoms over time so that the treatment regimens (medical and non-medical) can be refined as needed.

The objective data, physiological parameters and structured observations are integrated through algorithms to generate a rating of the following attentional characteristics and executive functions: level of arousal/alertness; cognitive tempo—impulsivity/reflectivity balance; vigilance; sustaining focus and filtering distractions; short-term working memory; response generation; complex problem solving; and/or self-monitoring/self-regulation.

These ratings can be used to enhance diagnostic accuracy of an evaluation for attention deficit disorder (ADD/ADHD) and other disorders of cognitive functioning. They can also serve as a baseline to tract the impact of various therapeutic interventions used to treat ADD/ADHD. Serial administration of the test/s of the present invention and its various iterations before, during, and after various interventions (e.g. medication, biofeedback, counseling strategies, etc.) generates comparative data in graphic, tabular, and other forms that can facilitate clinical decision-making. Similarly, serial administration of the test/s of the present invention and its various iterations makes it possible to track the development of attention and executive functioning over time and allows for comparison with the progression of these functions in neurotypical control populations. Another advantage or benefit is with the use of graphical images, it can be easily administered to a broad multilingual population across many ages from children to adults. The only pre-requisite is that the subjects understand the concept of “same/different”.

In one embodiment, the invention is a system for evaluating attentional ability, executive functions, and attention deficit disorders and other disorders of cognitive functioning, including an electronic touch screen display configured to display visual information via a graphical user interface, a processor configured to control the electronic touch screen display, wherein the electronic touch screen display is configured to display a set of indicia or images comprising at least one of the following: at least one identical matching pair of indicia or images and at least one indicia or images distinct from other indicia or images of the set of indicia or images, wherein the processor is configured to record an input to the system to a writable memory, wherein the input recorded to the writable memory comprises at least one of the following: a user's touching of both indicia or image of the at least one identical matching pair of indicia or images displayed on the electronic touch screen display and a user's touching of the at least one indicia or images distinct from other indicia or images of the set of indicia or images, wherein the input recorded to writable memory further comprises at least one of a time taken to touch an indicia or image, a number of correct matches, a number of incorrect matches, a demographic input regarding the user, the user's heart rate, the user's galvanic skin resistance, the user's eye movements, the user's facial expression, or other physiological input, wherein the processor is configured to generate a rating for at least one of the user's level of arousal, the user's level of alertness, the user's cognitive tempo, the user's vigilance, the user's short-term working memory, the user's response generation, the user's complex problem solving or the user's self monitoring based on the input recorded to writable data. In another embodiment the processor is configured to randomize the position of the set of indicia or images, and the processor is configured to randomize the composition of the set of indicia or images displayed chosen from a database of indicia or images. In another embodiment the processor is configured to randomize the number of indicia or images displayed, and the processor is configured to randomize the position of the set of indicia or images to be displayed chosen from a database of indicia or images. In another embodiment the invention further includes a camera configured to record a video of the user, the video is recorded onto the writable memory via the processor. In another embodiment the processor is configured to analyze said video to determine one of the user's facial expressions, the user's eye movements, and the user's emotional states. In another embodiment the processor is configured to analyze said video to determine changes in background light or movement. In another embodiment the processor is configured to analyze said video to determine changes in background light or movement, and wherein the processor is configured to provide instructions to the user if a predetermined level of movement or changes in background light is detected from the video. In another embodiment the invention further includes a speaker, where the processor is configured to output audio instructions via said speaker to the user. In another embodiment the invention includes a microphone, wherein said processor is configured to detect sounds from the microphone. In another embodiment the processor is configured to analyze said sounds via the microphone to determine a level of background noise, and where the processor is configured to provide instructions to the user if a predetermined level of background noise is detected from the microphone.

In one embodiment, the invention is a method of evaluating attentional ability, executive functions, and attention deficit disorders and other disorders of cognitive functioning, including the steps of providing an apparatus including an electronic touch screen display configured to display visual information via a graphical user interface, a processor configured to control the electronic touch screen display, wherein the electronic touch screen display is configured to display a set of indicia or images comprising at least one of the following: at least one identical matching pair of indicia or images and at least one indicia or images distinct from other indicia or images of the set of indicia or images, wherein the processor is configured to record an input to the system to a writable memory; recording to writable memory at least one of a time taken to touch both indicia or image of the at least one identical matching pair of indicia or images, a time taken to touch at least one indicia or images distinct from other indicia or images of the set of indicia or images, a number of correct matches, a number of incorrect matches, a demographic input regarding the user, the user's heart rate, the user's galvanic skin resistance, the user's eye movements, the user's facial expression, or other physiological input, and generating a rating for at least one of the user's level of arousal, the user's level of alertness, the user's cognitive tempo, the user's vigilance, the user's short-term working memory, the user's response generation, the user's complex problem solving or the user's self monitoring based on the input recorded to writable data. In another embodiment the processor is configured to randomize the position of indicia or images of the set of indicia or images, and wherein the processor is configured to randomize the set of indicia or images selected to be displayed chosen from a database of indicia or images. In another embodiment the processor is configured to randomize the number indicia or images displayed, and where the processor is configured to randomize the position of the set of indicia or images selected to be displayed chosen from a database of indicia or images. In another embodiment the apparatus includes a camera configured to record a video of the user, wherein said video is recorded onto the writable memory via the processor. In another embodiment the processor is configured to analyze said video to determine one of the user's facial expressions, the user's eye movements, and the user's emotional states. In another embodiment the processor is configured to analyze said video to determine changes in background light or movement. In another embodiment the processor is configured to analyze said video to determine changes in background light or movement, and wherein the processor is configured to provide instructions to the user if a predetermined level of movement or changes in background light is detected from the video. In another embodiment the apparatus includes a speaker, wherein said processor is configured to output audio instructions via said speaker to the user. In another embodiment the apparatus includes a microphone, where said processor is configured to detect sounds from the microphone. In another embodiment the processor is configured to analyze said sounds via the microphone to determine a level of background noise, and where the processor is configured to provide instructions to the user if a predetermined level of background noise is detected from the microphone.

### BRIEF DESCRIPTION OF THE DRAWINGS

FIG. 1 Shows a tablet computer configured to conduct the diagnostic method of the present invention;

FIG. 2 Shows an initial screen on a tablet computer;

FIG. 3 Shows another initial screen on a tablet computer for entering identifying information of a subject;

FIG. 4 Shows another initial screen on a tablet computer for selecting or adding a new subject;

FIG. 5 Shows a subject screen on a tablet computer;

FIG. 6 Shows a medication screen on a tablet computer;

FIG. 7 Shows a new test screen on a tablet computer;

FIG. 8 Shows another screen on a tablet computer providing instructions for taking a test;

FIG. 9 Shows a screen displaying a first example plate;

FIG. 10 Shows a screen displaying a second example plate;

FIG. 11 Shows a starting the formal test screen on a tablet computer;

FIG. 12 Shows a user selecting two images on a search section plate displayed on a tablet computer;

FIG. 13 Shows a table presenting the number of distinctive features which differentiate the two identical faces from the remainder of the field for each plate in a search section;

FIG. 14 Shows an example of a scan section plate displayed on a tablet computer;

FIG. 15 Shows a table presenting the number of distinctive features which differentiate the two identical faces from the remainder of the field for each plate in a scan section;

FIG. 16 Shows an example of an extended field search section plate displayed on a tablet computer; and

FIG. 17 Shows an example of a generated report showing results of a test of the present invention.

### BRIEF DESCRIPTION OF THE INVENTION

The present invention provides for a diagnostic method and apparatus for screening and assisting in diagnosing attention deficit hyperactivity disorder (ADD/ADHD) in a person. In many embodiments the invention includes a visual problem solving test involving distinctive feature analysis.

These and other features and characteristics of the present invention, as well as the methods of operation and functions of the related elements of structures and the combination of parts and economies of manufacture, will become more apparent upon consideration of the following description with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification, the singular form of “a”, “an”, and “the” include plural referents unless the context clearly dictates otherwise.

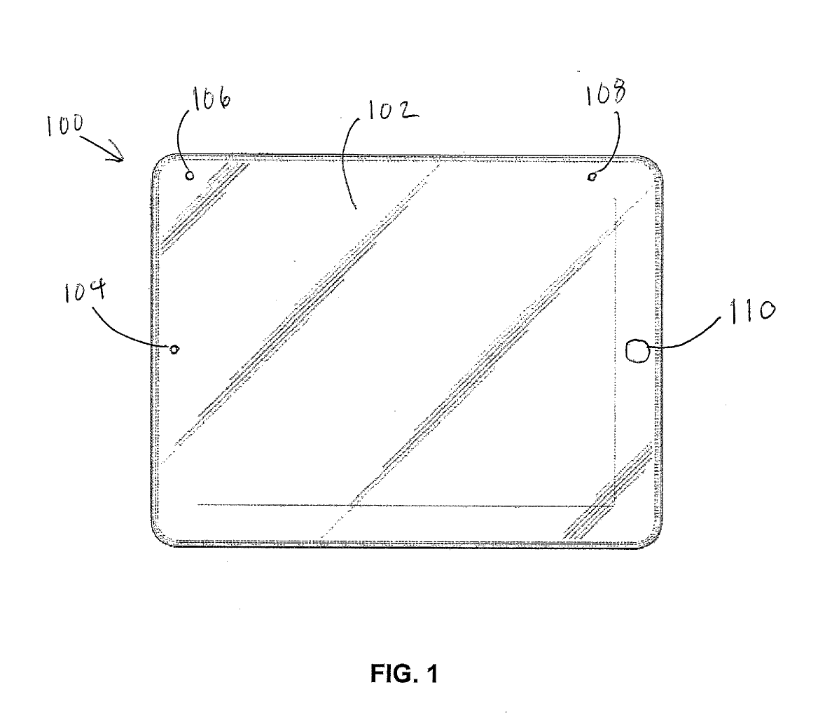

Referring generally to FIG. 1, a tablet computer 100 configured to conduct the diagnostic method of the invention is shown. The tablet computer 100 includes a multi-touch screen 102, digital video camera 104, speaker 106, microphone 108, and home/power button 110. The tablet computer 100 also includes a processor (not shown), writable memory (not shown), and an electrical power supply (not shown). In some embodiments of the present invention the tablet computer 100 may also include a wireless communications adapter for connecting to the internet. It should be understood that some actions or the present invention could take place remotely via a cloud server as opposed to internally with the tablet computer's processor and writable memory. It should also be understood that heart rate monitors or electrodes external to the tablet computer 100 may be used to determine a subject's heart rate and galvanic skin resistance, respectfully.

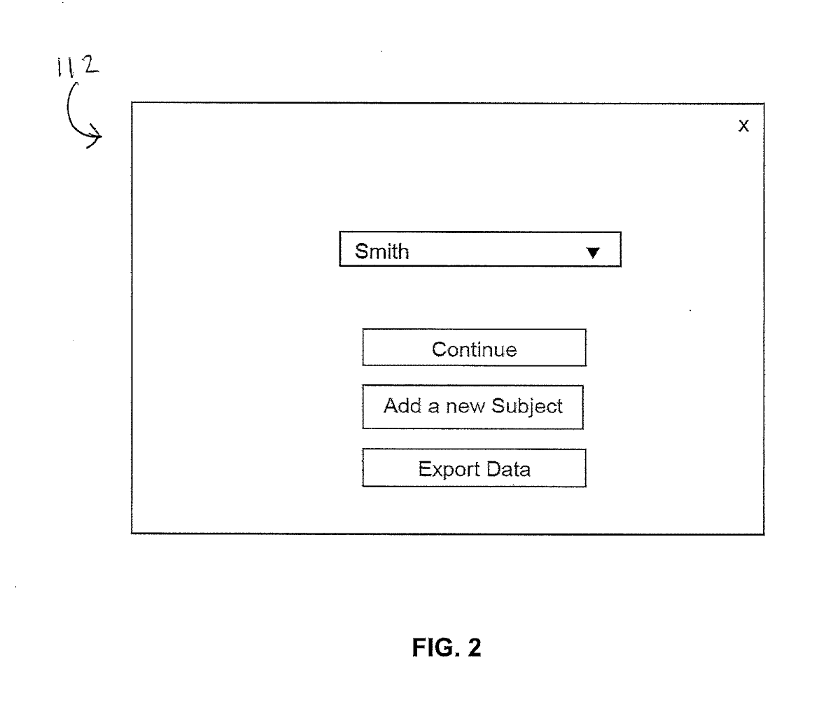

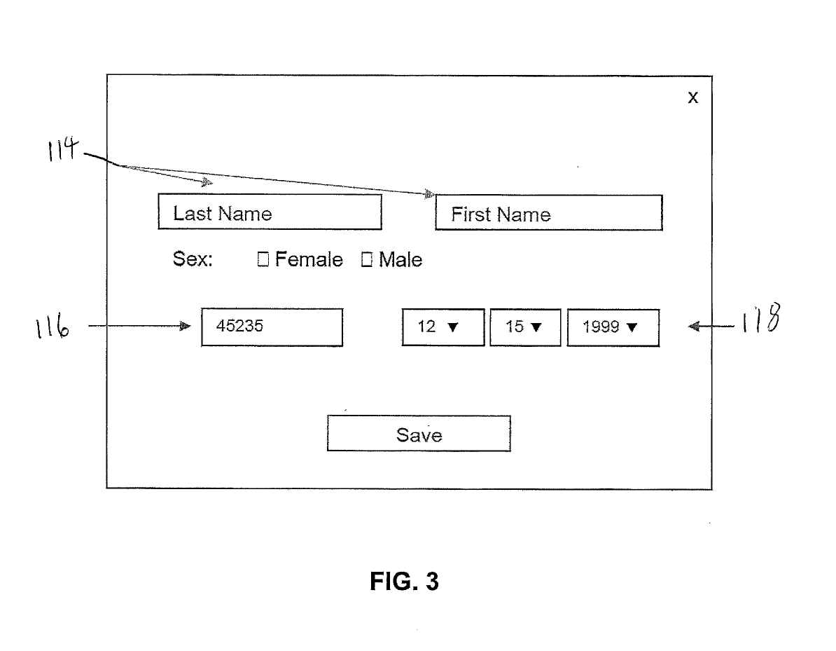

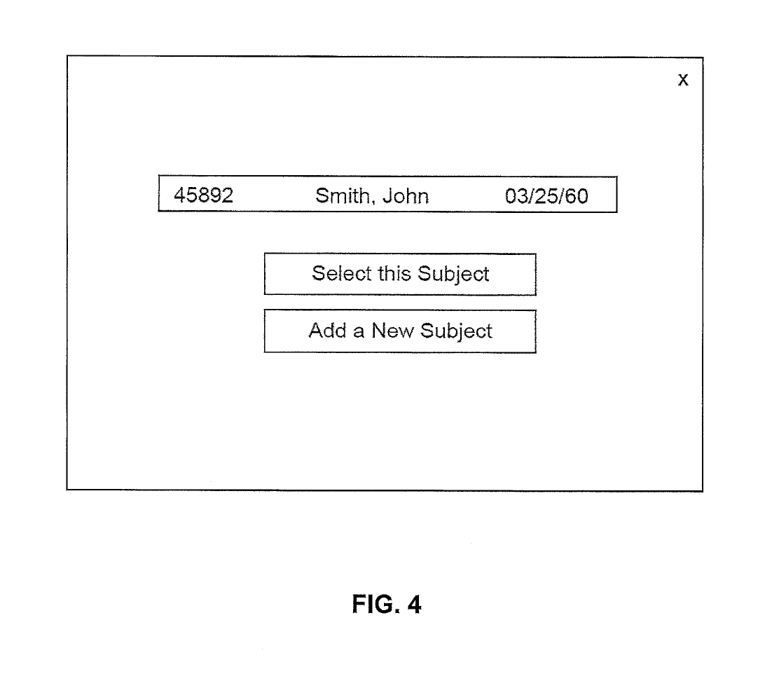

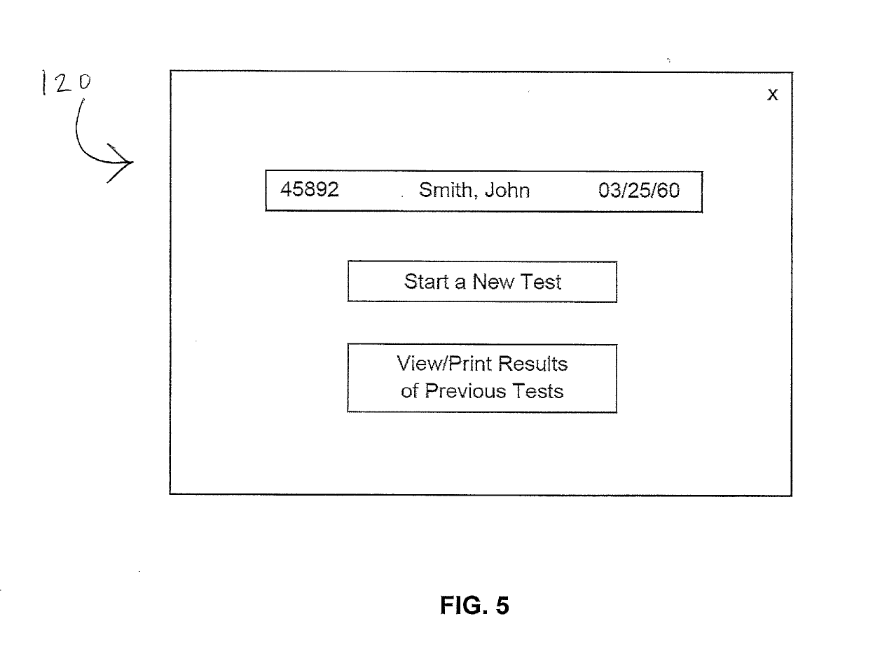

Referring generally to FIGS. 2-14, which show examples of screens shown to users/subjects taking the test of the present invention on the tablet computer 100. Referring to FIGS. 2-4 specifically, the initial screen is Select/Add a subject screen 112. Users will either start typing in the first few letters of their name 114 or record number 116 to search for a subject already in the system. The system will have the ability to search and recognize records with the same spelling of the subject's last name and present to Users one or more subjects in the system for Users to then highlight and click/touch “Continue”. Users will need to complete all entry boxes 118 before being allowed to click/touch “Save”. If the subject's first name, last name and date of birth are the same of another subject in the system, a message will appear as shown in FIG. 4. Referring to FIG. 4, clicking/touching “Select this subject” will take Users to the subject screen 120, shown in FIG. 5. Clicking/touching “Add a New subject” will take Users back to a new blank subject screen 112 to re-attempt adding a new/different subject's information. Once Users select, add, or save a new subject, they will then progress to the subject screen 120 to select their next choice of action.

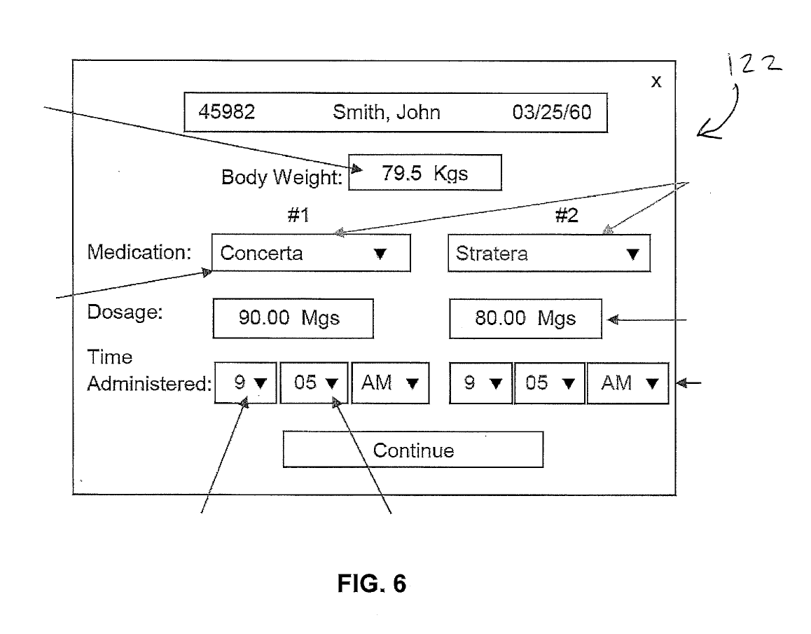

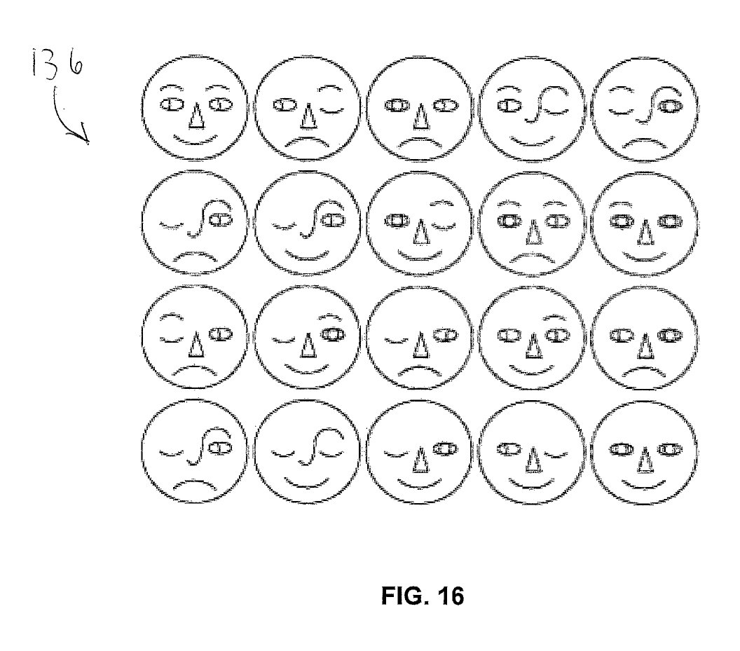

Referring to FIG. 5, upon clicking/touching “Start a New Test”, Users will be taken to the medication screen 122, shown in FIG. 6. Upon clicking/touching “View/Print Results of Previous Tests”, Users will be taken to the “Printing the Results” of a subject's tests, where a report as shown in FIG. 16 may be viewed or printed, and is detailed further below.

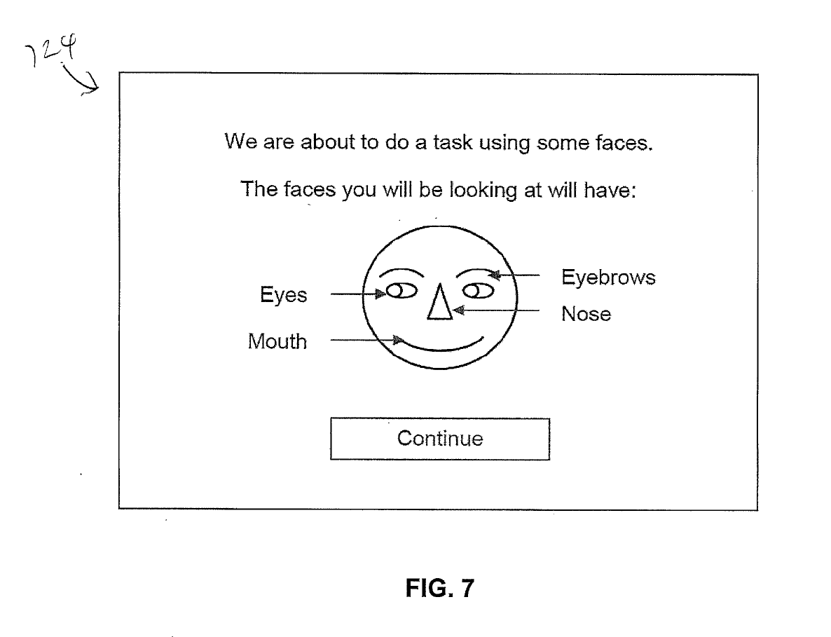

Referring to FIG. 6, if the subject is performing the test under the influence of a prescribed psychotropic medication, the name, dosage, and time of administration will be filled in on the Medication Screen 122. No medication is required to be entered on the medication screen 122. If no medication is entered, then tapping “Continue” will take the user to the starting a new test screen 124, shown in FIG. 7.

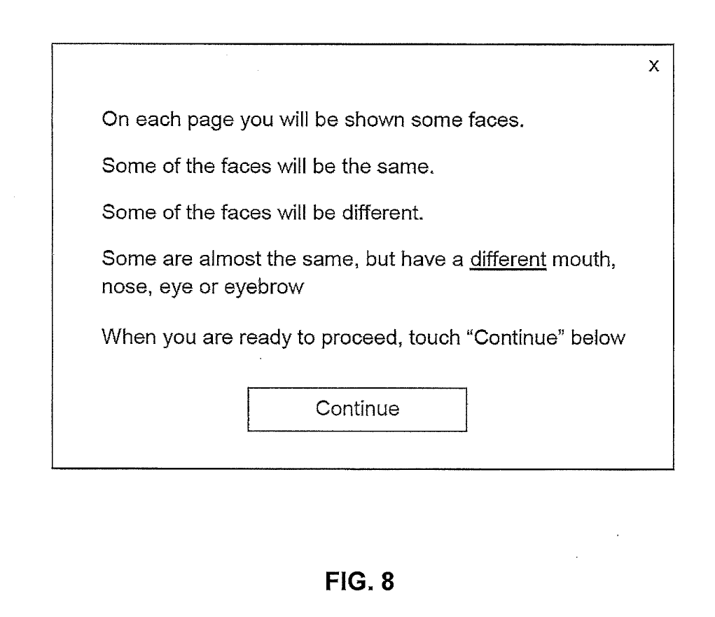

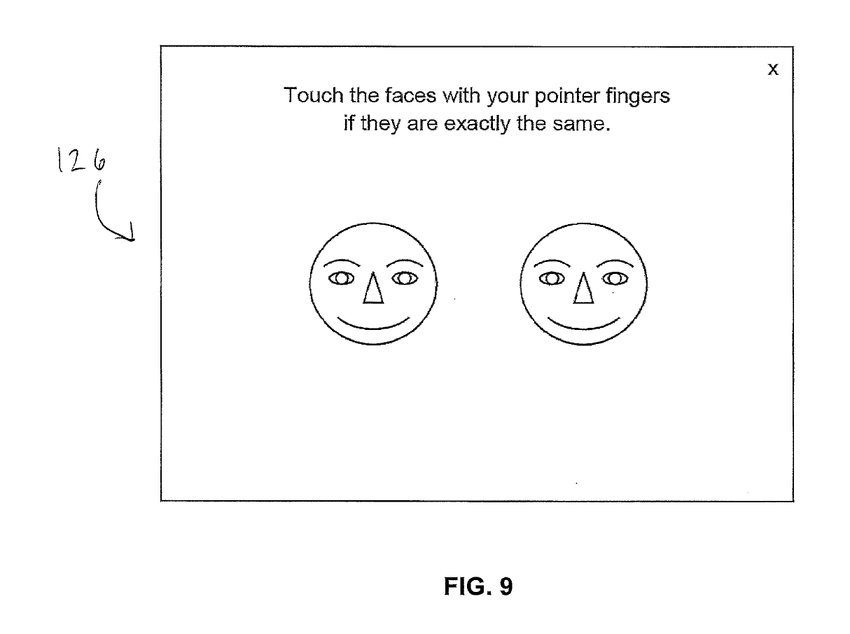

Referring to FIGS. 7 & 8, the starting a new test screen 124 starts the process of providing the subjects and/or users with a brief description of the test read from a script. In this example the images or indicia are simple illustrated faces and distinctive features of these faces include various eyes, eyebrows, noses, and mouths. In some embodiments of the present invention a graphical avatar displayed on multi-touch screen 102 provides the instructions, guides the user, and potentially interacts with the user as part of the testing process. The graphical avatar may provide the instructions via text instructions and/or audio instructions in any number of different languages. The user taps “Continue” to progress to a screen of further instructions shown in FIG. 8. The user then taps “Continue” shown in FIG. 8 to progress to the next screen shown in FIG. 9.

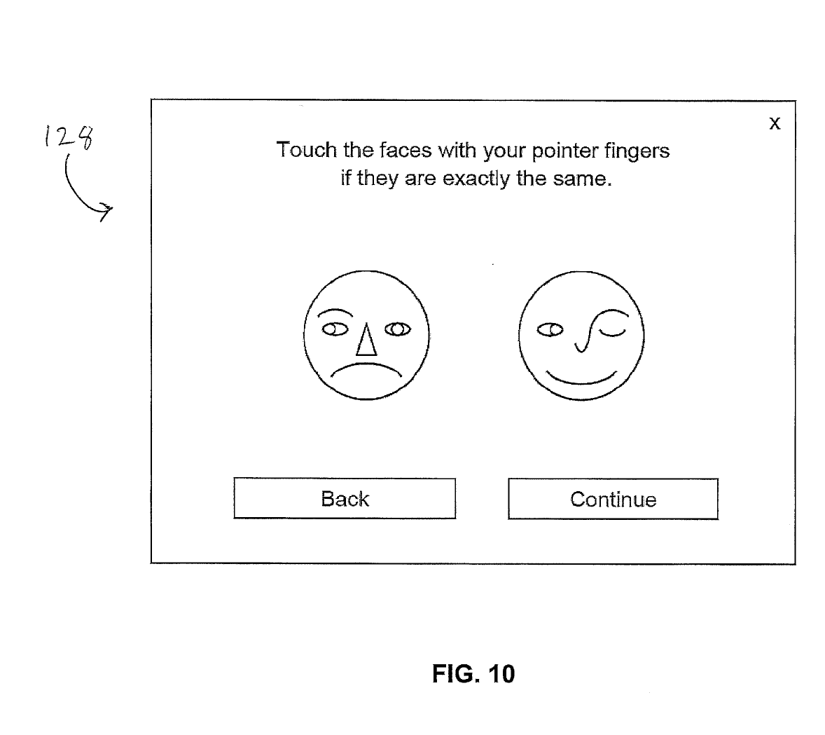

Referring to FIGS. 9 & 10, this section of the test serves two purposes. First, it provides a context in which to instruct the subject as to the nature of the task and materials involved. In addition, it provides an opportunity to test whether the subject has a meaningful grasp of one of the prerequisites for successfully mastering the concept of same/different. FIG. 9 shows a first example plate 126 with two identical images, as mentioned above in this example the images are faces. The user is instructed to touch both faces at exactly the same time if they are exactly the same. Whether one, both or neither of the faces are selected, the screen will transition after seven seconds to the next plate, second example plate 128, shown in FIG. 10. On the screen of second example plate 128 with two different faces the user is instructed to touch both faces at exactly the same time if they are exactly the same. If the subject does not answer both of these example plates correctly, then they are sent to the beginning of the test at the new test screen 124. They are sent back to the beginning, since it appears they do not grasp the concept of “same/different” and are instructed again in the hopes they will grasp this concept with repeated instruction.

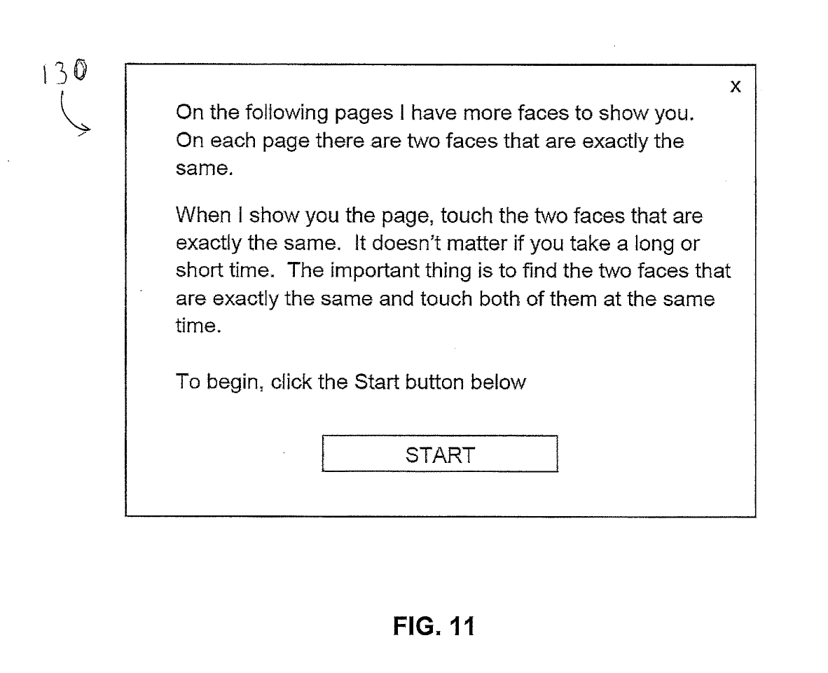

Referring to FIG. 11, the starting the formal test screen 130 includes a brief description of the formal test. Specifically, starting the formal test screen 130 explains that the subject is to touch the two faces that are exactly the same. Once “START” is tapped the tablet computer 100 will record the date and time in the subject's record as the beginning of recording the test session's data. The subject is then taken to the search section of plates.

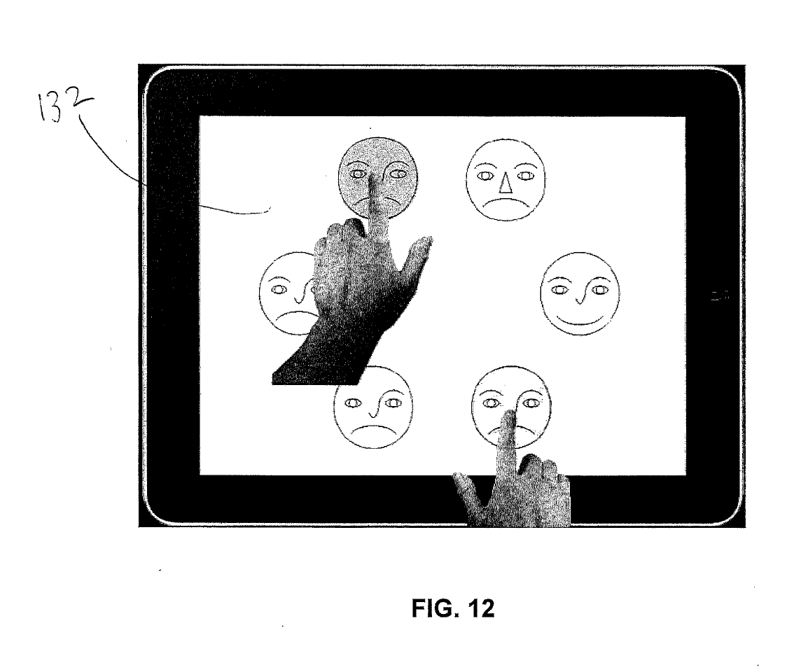

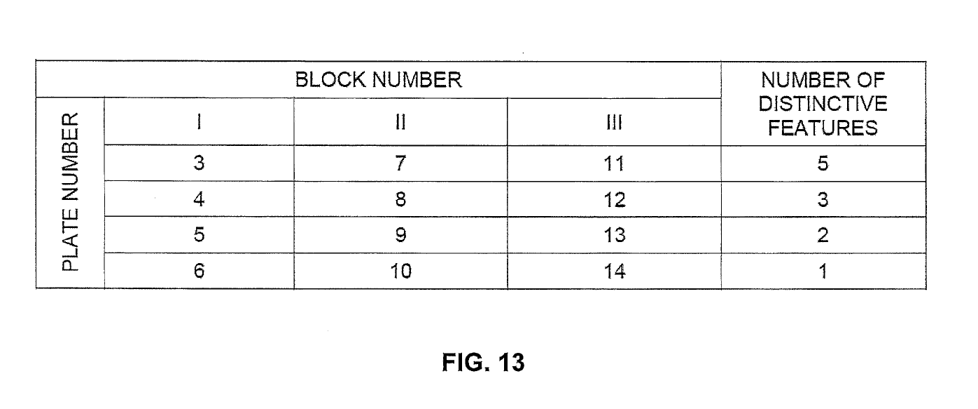

FIG. 12 shows a subject selecting two faces on a search section plate 132. In this embodiment the search section includes plates numbers 3-14, faces in each of these plates in are arranged in a circular fashion, each image being equidistant from a center point. In this section, the subject is required to find the two identical images. Performance on each plate generates a correct/incorrect score and a response time, in addition to a recording of the specific items selected. In addition to mobilizing a need for sustained, focused attention and executive functioning, this section requires that the subject spontaneously mobilize various executive functions in order to develop a problem solving strategy and apply it to a novel situation in order to perform at maximum efficiency. This section consists of three blocks of items: Block I: field of three images (Plates 3-6); Block II: field of six images (Plates 7-10); and Block III: field of ten images (Plates 11-14). Within each of these blocks of plates, the number of distinctive features which differentiate the two identical faces from the remainder of the field progressively decrease as illustrated in the table shown in FIG. 13. After completing plates numbers 3-14, the subject is taken to the next set of plates, the scan section.

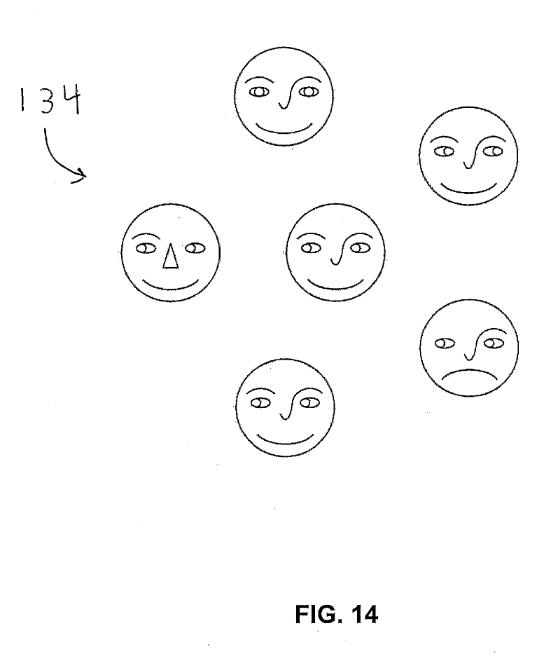

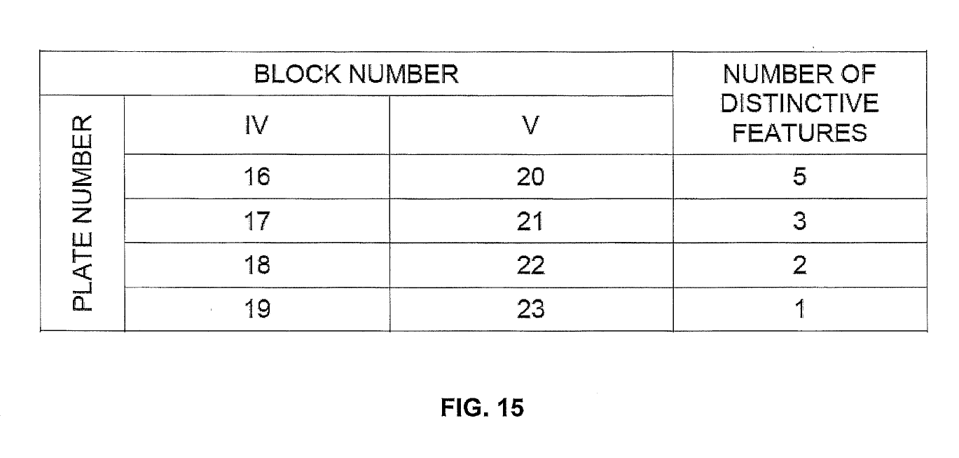

Referring to FIG. 14, the scan section of the test includes plates numbers 16-23. FIG. 16 shows an example of a scan section plate 134. Images on each of these plates are arranged with one target image in the center and the remaining images distributed about this central image in an equidistant fashion. In this section, a specific strategy is imposed on the subject. One of the pair of identical images (the central target image) is identified for the subject and he/she is requested to find a match from the remainder of the field. Similar data are obtained for each item as noted above. With the imposition of a specific strategy and its placement after the Search Section, this section provides an optimal “window” to observe intrinsic attentional characteristics such as cognitive tempo, filtering, and vigilance and certain executive functions. As in the search section, the number of distinctive features which differentiate the target image and its matched pair from the remainder of the field progressively decreases as illustrated in the table of FIG. 15. After completing plates numbers 16-23, the subject is taken to the next set of plates, the extended field search section.

Referring to FIG. 16, the extended field search section includes plates numbers 15 and 24. FIG. 16 shows an example of an extended field search plate 136. These two plates are made up of 20 images each including six matched pairs that are randomly distributed in the total field subjects are not informed that these plates are identical. One of these plates (15) follows immediately after Block III of the Search Section and the other (24) follows immediately after Block V of the Scan Section. These specific placements are employed to provide a means to determine if a subject learned from the imposition of a specific strategy during the scan section and was able to generalize as measured by a more efficient performance on the repeat trial on an identical task.

The embodiments described above can be modified in a variety of ways to create various iterations of the test. This may include some combination of the following modifications:

A. Altering the number or type of images to be selected by the subject on a given plate or within sections (ex. Search, Scan, Extended Field Search, etc.):
a. More than two identical images; and/or
b. More than two different images.

B. Altering the number of images and the progression of the number of distinctive features within a given section or block within a section:
a. Increasing numbers of images and increasing number of distinctive features;
b. Increasing numbers of images and decreasing number of distinctive features;
c. Increasing numbers of images and keeping static the number of distinctive features;
d. Decreasing numbers of images and increasing number of distinctive features;
e. Decreasing numbers of images and decreasing number of distinctive features;
f. Decreasing numbers of images and keeping static the number of distinctive features;
g. Keeping static the numbers of images and keeping static the number of distinctive features;
h. Keeping static the numbers of images and increasing number of distinctive features; and/or
i. Keeping static the numbers of images and decreasing number of distinctive features.

C. Expanding or contracting the number of blocks and their order of presentation.

D. Expanding or contracting the number of images and/or the number of matches on at least one plate.

E. Altering plates to be non-identical or dissimilar with respect to the number of images, the total number of matched pairs/matches, and/or the number of images that are identical that need to be selected by the subject.

F. Altering the types of images within a given test format from plate-to-plate, block-to-block, or section-to-section.

For a given test configuration, multiple random arrangements of positions on a plate are possible. The randomization of the number and type of images and distinctive features across the plates of the search section, scan section, and/or the extended filed search section could be conducted via the processor of the tablet computer 100 or via a cloud server's processor.

It is also envisioned that instead of having the user search for matching pairs of images or indicia on a plate, they could search for at least one image that is different in a field of mostly matching images. This arrangement could be in the alternative or in addition to searching for matching pairs of images or indicia on a plate. The automatic random feature of the present invention allows for more reliable serial testing of a user, since the user will not be able to memorize the randomly generated plates.

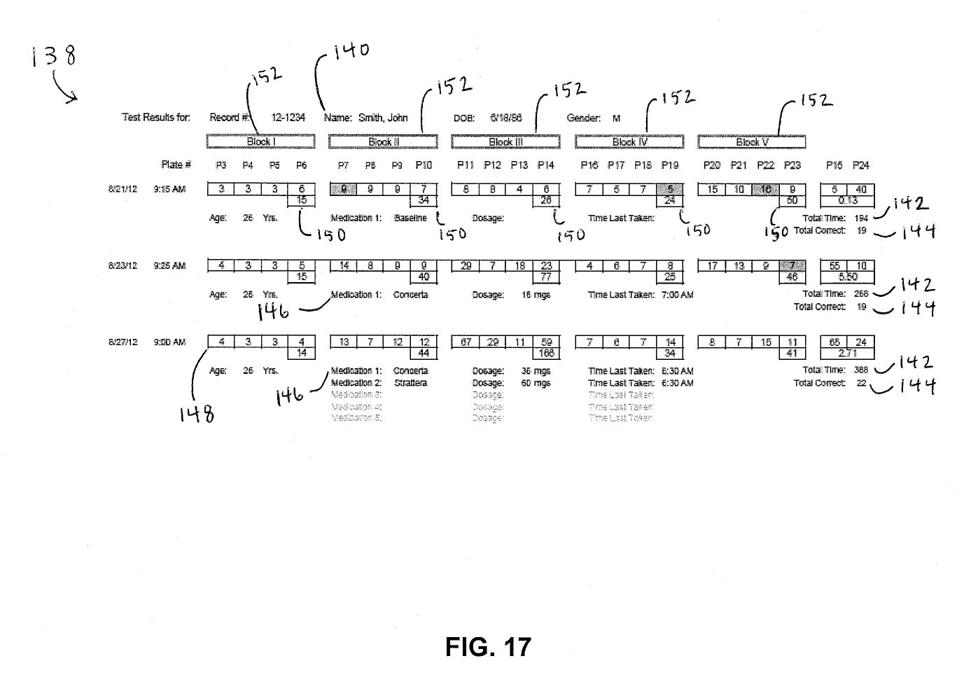

Referring to FIG. 17, once the test has been completed the recorded data and the results of the analysis of that data can be exported to another application or device via options in the application. The user is also able to view and/or print a report 138 of the results of the test as shown in FIG. 17. The example report 138 includes the user's identifying information 140, the total time to take each test, the total correct for each test 144, and medication information for the user 146. The report 138 also charts the time taken for each plate in first boxes 148, and totals them in second boxes 150. First boxes 148 are shaded if the user answered incorrectly on that plate. Third boxes 152 indicate in which block each plates resides.

Although the invention has been described in detail for the purpose of illustration based on what is currently considered to be the most practical and preferred embodiments of the application, it is to be understood that such detail is solely for that purpose and that the invention is not limited to the described embodiment. To the contrary, it is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A system for evaluating attentional ability, executive functions, and attention deficit disorders and other disorders of cognitive functioning, comprising:
an electronic touch screen display configured to display visual information via a graphical user interface,
a processor configured to control the electronic touch screen display,
wherein the electronic touch screen display is configured to display a set of indicia or images comprising at least one of the following: at least one identical matching pair of indicia or images and at least one indicia or images distinct from other indicia or images of the set of indicia or images, wherein the processor is configured to record an input to the system to a writable memory, wherein the input recorded to the writable memory comprises at least one of the following: a user's touching of both indicia or image of the at least one identical matching pair of indicia or images displayed on the electronic touch screen display and a user's touching of the at least one indicia or images distinct from other indicia or images of the set of indicia or images, wherein the input recorded to writable memory further comprises at least one of a time taken to touch an indicia or image, a number of correct matches, a number of incorrect matches, a demographic input regarding the user, the user's heart rate, the user's galvanic skin resistance, the user's eye movements, the user's facial expression, or other physiological input, wherein the processor is configured to generate a rating for at least one of the user's level of arousal, the user's level of alertness, the user's cognitive tempo, the user's vigilance, the user's short-term working memory, the user's response generation, the user's complex problem solving or the user's self monitoring based on the input recorded to writable data.

2. The system of claim 1, wherein the processor is configured to randomize the position of the set of indicia or images, and
wherein the processor is configured to randomize the composition of the set of indicia or images displayed chosen from a database of indicia or images.

3. The system of claim 1, wherein the processor is configured to randomize the number of indicia or images displayed, and
wherein the processor is configured to randomize the position of the set of indicia or images to be displayed chosen from a database of indicia or images.

4. The system of claim 1, further comprising a camera configured to record a video of the user, wherein said video is recorded onto the writable memory via the processor.

5. The system of claim 4, wherein the processor is configured to analyze said video to determine one of the user's facial expressions, the user's eye movements, and the user's emotional states.

6. The system of claim 4, wherein the processor is configured to analyze said video to determine changes in background light or movement.

7. The system of claim 4, wherein the processor is configured to analyze said video to determine changes in background light or movement, and wherein the processor is configured to provide instructions to the user if a predetermined level of movement or changes in background light is detected from the video.

8. The system of claim 1, further comprising a speaker, wherein said processor is configured to output audio instructions via said speaker to the user.

9. The system of claim 1, further comprising a microphone, wherein said processor is configured to detect sounds from the microphone.

10. The system of claim 9, wherein the processor is configured to analyze said sounds via the microphone to determine a level of background noise, and wherein the processor is configured to provide instructions to the user if a predetermined level of background noise is detected from the microphone.

11. A method of evaluating attentional ability, executive functions, and attention deficit disorders and other disorders of cognitive functioning, comprising the steps of:
providing an apparatus comprising: an electronic touch screen display configured to display visual information via a graphical user interface, a processor configured to control the electronic touch screen display, wherein the electronic touch screen display is configured to display a set of indicia or images comprising at least one of the following: at least one identical matching pair of indicia or images and at least one indicia or images distinct from other indicia or images of the set of indicia or images, wherein the processor is configured to record an input to the system to a writable memory;
recording to writable memory at least one of a time taken to touch both indicia or image of the at least one identical matching pair of indicia or images, a time taken to touch at least one indicia or images distinct from other indicia or images of the set of indicia or images, a number of correct matches, a number of incorrect matches, a demographic input regarding the user, the user's heart rate, the user's galvanic skin resistance, the user's eye movements, the user's facial expression, or other physiological input, and generating a rating for at least one of the user's level of arousal, the user's level of alertness, the user's cognitive tempo, the user's vigilance, the user's short-term working memory, the user's response generation, the user's complex problem solving or the user's self monitoring based on the input recorded to writable data.

12. The method of claim 11, wherein the processor is configured to randomize the position of indicia or images of the set of indicia or images, and wherein the processor is configured to randomize the set of indicia or images selected to be displayed chosen from a database of indicia or images.

13. The method of claim 11, wherein the processor is configured to randomize the number indicia or images displayed, and
wherein the processor is configured to randomize the position of the set of indicia or images selected to be displayed chosen from a database of indicia or images.

14. The method of claim 1, wherein the apparatus further comprises a camera configured to record a video of the user, wherein said video is recorded onto the writable memory via the processor.

15. The method of claim 14, wherein the processor is configured to analyze said video to determine one of the user's facial expressions, the user's eye movements, and the user's emotional states.

16. The method of claim 14, wherein the processor is configured to analyze said video to determine changes in background light or movement.

17. The method of claim 14, wherein the processor is configured to analyze said video to determine changes in background light or movement, and wherein the processor is configured to provide instructions to the user if a predetermined level of movement or changes in background light is detected from the video.

18. The method of claim 1, wherein the apparatus further comprises a speaker, wherein said processor is configured to output audio instructions via said speaker to the user.

19. The method of claim 1, wherein the apparatus further comprises a microphone, wherein said processor is configured to detect sounds from the microphone.

20. The method of claim 19, wherein the processor is configured to analyze said sounds via the microphone to determine a level of background noise, and wherein the processor is configured to provide instructions to the user if a predetermined level of background noise is detected from the microphone.
